(19) United States
(12) Patent Application Publication
(10) Pub. No.: US 2022/0202643 A1
(43) Pub. Date: Jun. 30, 2022

(54) HANDS-FREE NECK AND SHOULDER MASSAGER

(71) Applicant: NEKTECK, INC., Anaheim, CA (US)

(72) Inventor: JINLONG LIN, Anaheim, CA (US)

(21) Appl. No.: 17/189,861

(22) Filed: Mar. 2, 2021

(30) Foreign Application Priority Data
Dec. 31, 2020 (CN) 202011632861.3

(51) Int. Cl. A61H 15/02; A61H 15/00

(52) U.S. Cl. CPC A61H 15/02; A61H 15/0078; A61H 2201/1215; A61H 2201/1614; A61H 2201/0221; A61H 2205/04; A61H 2205/062; A61H 2201/0207

(57) ABSTRACT

A massager with heating features includes at least one massage head, one heating lamp, an upper housing, a carriage, a motor, one set of gear assembly, and a lower housing.

### FIELD

The subject matter herein generally relates to a neck and shoulder massager, and a more particularly, to a shoulder massager in which two built-in massage heads rotate while the massager is placed on the user's shoulder.

### BACKGROUND

Massagers that relax muscles of a body, for example, neck and shoulder areas, are popular among people who spend a lot of time in a same position (e.g., working in front of a computer or sitting in the driver seat). Throughout development of massagers, those with heating features become desired because heat allows better circulation, which induces relaxation. However, conventional heat massagers often require a complex structure and elaborate wiring management. The structural complexity results in high cost.

In patent U.S. Pat. No. 7,722,553 B2, HoMedics Inc. describes a massage apparatus that provides heat and massage features simultaneously, which uses conductive brushes to maintain electrical communication during rotation. However, the wear and tear of the conductive brushes result in high replacement cost.

In one or more implementations, the present disclosure provides an easy solution for a low-cost heat massager. Therefore, there is room for improvement within the art.

### SUMMARY OF THE INVENTION

A massager comprises at least one massaging member comprising one or more massage heads; a housing; a motor disposed in the housing; at least one gear assembly operably connected to the motor for providing rotation to the massaging member; at least one substrate attached to a top portion of the housing; and one or more heating elements mounted to the substrate.

The housing comprises an upper housing and a lower housing, the upper housing has a cylindrical shaped portion and a plurality of slits on the cylindrical shaped portion, and the motor is received in the cylindrical shaped portion.

The at least one gear assembly comprises an inner gear and an outer gear. The at least one substrate can be a printed circuit board or the like. The one or more heating elements can be infrared lamps or the like.

### BRIEF DESCRIPTION OF THE DRAWINGS

Implementations of the present technology will now be described, by way of example only, with reference to the attached figures.

FIG. 1 is a perspective view of an embodiment of a massager in accordance with the present disclosure.

FIG. 2 is another perspective view of the massager in FIG. 1.

FIG. 3 is a partially exploded view of the massager in FIG. 1.

FIG. 4 is a rear side view of two massage members of the massager in Fig. 3.

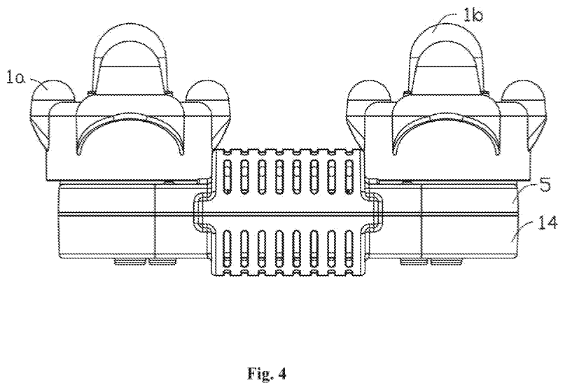

FIG. 5 is a top plan view of two massage members of the massager in FIG. 3.

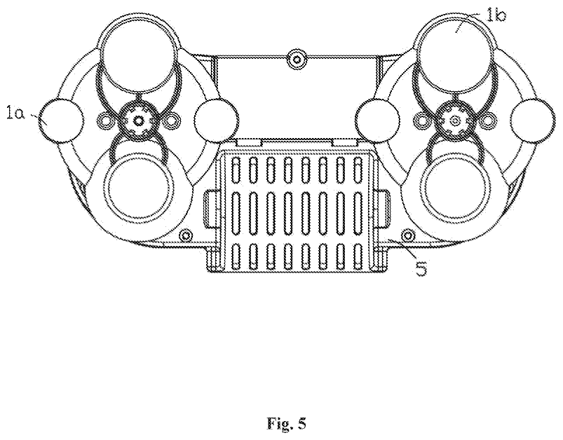

FIG. 6 is a left side view of two massage members of the massager in FIG.

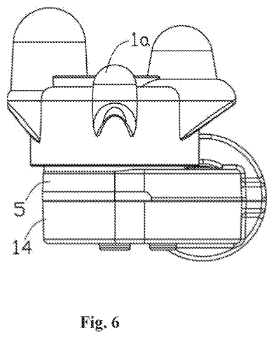

3.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, it will be understood by those of ordinary skill in the art that the embodiments described herein can be practiced without these specific details. In other instances, methods, procedures, and components have not been described in detail so as not to obscure the related relevant feature being described. Also, the description is not to be considered as limiting the scope of the embodiments described herein. The drawings are not necessarily to scale, and the proportions of certain parts may be exaggerated to illustrate details and features of the present disclosure better. The disclosure is illustrated by way of example and not by way of limitation in the figures of the accompanying drawings, in which like references indicate similar elements. It should be noted that references to “an” or “one” embodiment in this disclosure are not necessarily to the same embodiment, and such references mean “at least one.”

The term “comprising” when utilized, means “including, but not necessarily limited to” specifically indicates open-ended inclusion or membership in the so-described combination, group, series, and the like.

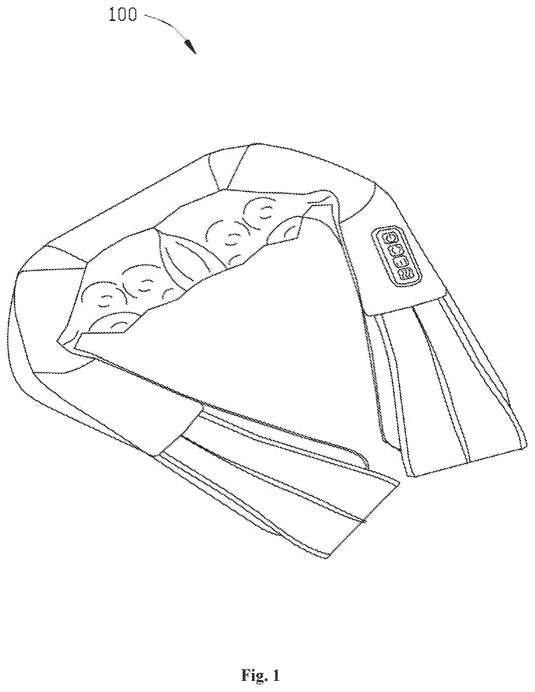

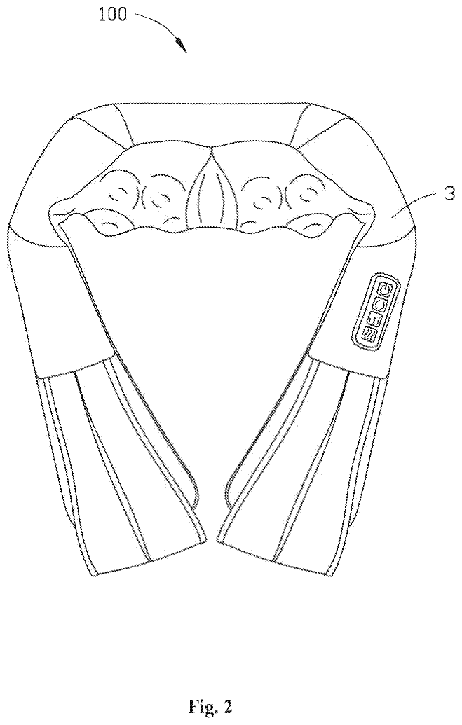

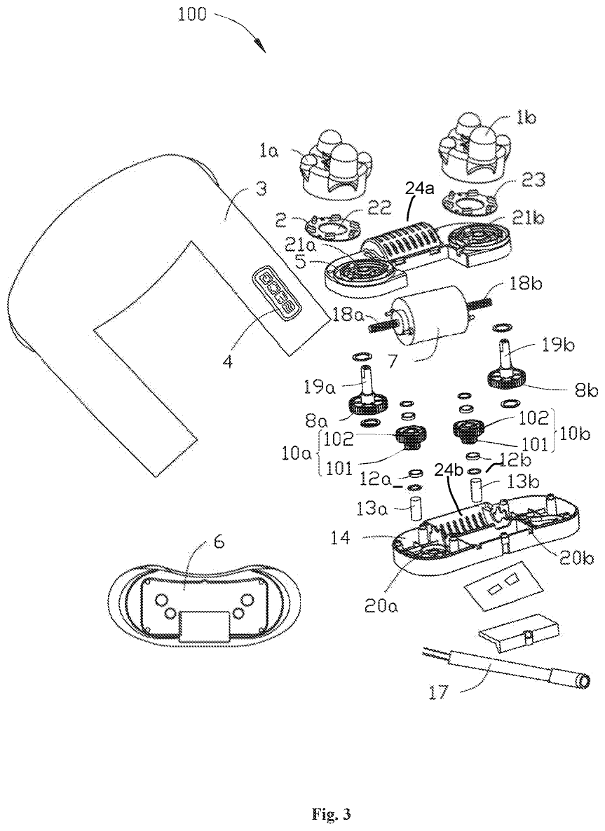

FIGS. 1-3 illustrate a massager 100 according to one embodiment of the present disclosure. In one or more implementations of the present disclosure, the massager 100 has two massage heads 1a and 1b, an upper housing 5, a carriage 6, a motor 7, and a lower housing 14, which are disposed inside an outer skin 3. Each of two Printed Circuit Boards (PCBs) 2 is attached to each of the two massage heads 1a and 1b, respectively. A heating lamp 23, for example a neon lamp, is attached on each of the two PCBs 2. The two massage heads 1a and 1b and the PCBs 2 are mounted on an outer surface (e.g., top portion) of the upper housing 5. Each massage head 1a (1b) is rotatable with respect to the upper housing 5. The upper housing 5 and the lower housing 14 are received in an interior space of the carriage 6. A main PCB (shown but not labeled) is in a cable cover (shown but not labeled), which is outside the housings 5, 14 and in the carriage 6. A jack 17 extends out of the outer skin 3.

In one or more implementations of the present disclosure, the motor 7, outer gears 8a and 8b, and inner gears 10a and 10b are received between the upper housing 5 and the lower housing 14. The motor 7 has a worm gear 18a and a worm gear 18b on opposite sides of the motor 7. The outer gear 8a engages with the worm gear 18a, and the outer gear 8b engages with the worm gear 18b. The inner gear 10a engages with the corresponding outer gear 8a, and the inner gear 10b engages with the corresponding outer gear 8b. The lower housing 14 has two first openings 20a and 20b formed on two sides of the lower housing 14, respectively. A shaft 13a is coupled to the first opening 20a, and a shaft 13b is coupled to the first opening 20b. The inner gear 10a is mounted on the shaft 13a through one or more washers 12a, and the inner gear 10b is mounted on the shaft 13b through one or more washers 12b.

In one or more implementations of the present disclosure, the upper housing 5 has a truncated cylindrical shape and a plurality of slits 24a formed on the body of the upper housing 5. The lower housing 14 has a truncated cylindrical shape, and a plurality of slits 24b formed on the body of the lower housing 14. The truncated cylindrical shape of the upper housing 5 and the truncated cylindrical shape of the lower housing 14 form a cylindrical-shaped housing where the motor 7 is received, and the plurality of slits 24a and 24b of the upper housing 5 and the lower housing 14 may disseminate heat generated by the motor 7.

In one or more implementations of the present disclosure, the first opening 20a and the first opening 20b are disposed, respectively, on truncated and cylindrically-shaped portions on the two sides of the lower housing 14.

In one or more implementations of the present disclosure, the inner gears 10a and 10b are disposed on the two opposite sides of the motor 7, respectively. The worm gear 18a engages with the inner gear 10a, and the worm gear 18b engages with the inner gear 10b. Each of the inner gears 10a and 10b includes a lower gear 101 and an upper gear 102 connecting to the lower gear 101. A radius of the lower gear 101 is smaller than a radius of the upper gear 102. The lower gear 101 of the inner gear l0a engages with the worm gear 18a, and the upper gear 102 of the inner gear 10a engages with the outer gear 8a. The lower gear 101 of the inner gear 10b engages with the worm gear 18b, and the upper gear 102 of the inner gear 10b engages with the outer gear 8b. The outer gear 8a has a shaft 19a that extends out of the upper housing 5 through a second opening 21a of the upper housing 5. The outer gear 8b has a shaft 19b that extends out of the upper housing 5 through a second opening 21b of the upper housing 5.

In one or more implementations of the present disclosure, each of the PCB s 2 may have a ring shape and has a through hole 22 at a center of the PCB 2. One or more heating lamps 23 may be distributed in intervals on each of the PCBs 2 in order to evenly generate heat on the massage heads 1a and 1b. The through hole 22 of one of the two PCBs 2 may align with the shaft 19a. The massage head 1a is connected to an end of the shaft 19a away from the inner gear 10a via the through hole 22. The through hole 22 of another PCB 2 may align with the shaft 19b. The massage head lb is connected to an end of the shaft 19b away from the inner gear 10b via the through hole 22 of the other PCB 2. The massage heads 1a, 1b cover the two PCBs 2, respectively. The PCBs 2 may be attached to the upper housing 5 and in electric communication with the two massage heads 1a and 1b as well as the heating lamps 23 to control operations (e.g., motions of the two massage heads 1a, 1b and/or heating of lamps 23) of the massager 100.

In one or more implementations of the present disclosure, the outer skin 3 may include a controller 4 on the outer surface of the outer skin 3, and the controller 4 may be electrically connected to the PCBs 2 and the motor 7 to control operations of the massager 100. A user can turn on and off the motor 7 and heating lamps 23 through the controller 4.

When the motor 7 is activated, the motor 7 drives the two worm gears 18a and 18b to rotate, and the rotation of the two worm gears 18a and 18b results in the rotation of the two inner gears 10a and 10b, which in turn results in the rotation of the two outer gears 8a and 8b. At least one washer (shown but not explicitly labeled) may be coupled to each of the two outer gears 8a and 8b and the two inner gears 10a and 10b and rotate with the two outer gears 8a and 8b and the two inner gears 10a and 10b when the two outer gears 8a and 8b and the two inner gears 10a and 10b rotate. The rotations of the two outer gears 8a and 8b further drive rotation of the two massage heads 1a and 1b.

It is to be understood, even though information and advantages of the present embodiments have been set forth in the foregoing description, together with details of the structures and functions of the present embodiments, the disclosure is illustrative only; changes may be made in detail, especially in matters of shape, size, and arrangement of parts within the principles of the present embodiments to the full extent indicated by the plain meaning of the terms in which the appended claims are expressed.

What is claimed is:

1. A massager comprising:
at least one massaging member comprising one or more massage heads;
a housing;
a motor disposed in the housing;
at least one gear assembly operably connected to the motor for providing rotation to the massaging member;
at least one substrate attached to a top portion of the housing; and
one or more heating elements mounted to the substrate.

2. The massager according to claim 1, wherein the housing comprises an upper housing and a lower housing.

3. The massager according to claim 2, wherein the upper housing has a cylindrical shaped portion and a plurality of slits on the cylindrical shaped portion, and the motor is received in the cylindrical shaped portion.

4. The massager according to claim 3, wherein the at least one gear assembly comprises an inner gear and an outer gear.

5. The massager according to claim 4, wherein the at least one substrate is a printed circuit board.

6. The massager according to claim 5, wherein the one or more heating elements are infrared lamps.

7. A massager comprising:
two massaging members each comprising one or more massage heads;
a housing;
a motor disposed in the housing;
two gear assemblies operably connected to the motor for providing rotation of each of the massaging members;
two substrates attached to a top portion of the housing corresponding to each of the massaging members; and
one or more heating elements mounted to the each substrate.

8. The massager according to claim 7, wherein the housing comprises an upper housing and a lower housing.

9. The massager according to claim 7, wherein the upper housing has a cylindrical shaped portion and a plurality of slits on the cylindrical shaped portion, and the motor is received in the cylindrical shaped portion.

10. The massager according to claim 7, wherein the two gear assemblies each comprise an inner gear and an outer gear.

11. The massager according to claim 7, wherein the substrate is a printed circuit board.

12. The massager according to claim 7, wherein the one or more heating elements are infrared lamps.
